(19) United States
(12) Patent Application Publication
(10) Pub. No.: US 2022/0184456 A1
(43) Pub. Date: Jun. 16, 2022

(54) REMOTE TRAINER(S)-TRAINEE(S) ELECTRONIC EXERCISE SYSTEM

(71) Applicant: TREADLY, INC., Hackensack, NJ (US)

(72) Inventor: Ronen HEN, Hackensack, NJ (US)

(21) Appl. No.: 17/552,238

(22) Filed: Dec. 15, 2021

Related U.S. Application Data
(60) Provisional application No. 63/126,329, filed on Dec. 16, 2020.

(51) Int. Cl. A63B 24/00; A63B 71/06

(52) U.S. Cl. CPC A63B 24/0075; A63B 24/0062; A63B 2214/00; A63B 2225/50; A63B 2230/00; A63B 71/0622

(57) ABSTRACT

An apparatus and method for remotely training one or more trainees by one or more trainers, using a 1-to-many duplex video/audio session(s). The sessions can be “live” sessions providing real-time input and feedback between the respective parties. A smart remote “fitness device(s)’ at the trainee(s)' site with the assistance of coordinating “app” provide to the trainer(s), accurate assessment of the trainee(s)' performance. The trainer(s) may adjust the activity (e.g., treadmill, resistance, etc.) of the remote fitness device(s) for increased or decreased performance. Real-time feedback from the trainee(s)' systems is provided for real-time analysis by the trainer(s).

### CROSS-REFERENCE TO RELATED APPLICATION(S)

This application claims the benefit of U.S. Provisional Patent Application No. 63/126,329, titled “Remote Trainer(s)-Trainee(s) Electronic Exercise System,” filed Dec. 16, 2020, the contents of which are hereby incorporated by reference in its entirety.

### FIELD

This invention relates to an exercise remote training system. More particularly, it relates to a feedback providing exercise system between a remote trainer(s) and trainee(s), allowing real-time monitoring and individualized equipment adjustment for trainees.

### BACKGROUND

Typical exercise training coordinated with a trainer involves one or more trainees being coached in person in a common setting or alternatively via a video link or simply via viewing a prerecorded training session from the trainer. In all of these scenarios, the trainer is not aware of the actual performance metrics of the trainee nor is the trainee's equipment real-time adjustable by the trainer. While there may be scenarios where the trainee's performance data is forwarded to a central system, this information is not utilized during a real-time training session. Therefore, a real-time best-workout program tailored for that particular trainee during a workout cannot be accomplished.

In view of the above deficiencies, various systems and methods for increasing training efficiency for remotely coordinated training sessions is described.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, a system for remote training trainees, utilizing a live trainer is provided, comprising: at least one smart fitness device, having at least a near range communication capability and a long-range communication capability, the long-range communication capability providing communication to a network cloud; at least one smart device, in proximity to the at least one smart fitness device, having a near range communication capability to the at least one smart fitness device and a long range communication ability to the network cloud; an exercise management App running on the at least one smart fitness device and smart device; and a server in the network Cloud, communicating with the App and managing other Apps on the at least one smart fitness device and smart device; wherein a Trainer using the App on his at least one smart fitness device and smart device is able to real-time monitor a metric of a remote Trainee training on her smart fitness device via the Trainee's smart fitness device's App or the Trainee's smart device's App, and alter an operational parameter of the Trainee's smart fitness device.

In another aspect of the disclosed embodiments, the system above is provided, wherein at least one of the smart fitness device(s) and smart device(s) communicates information to each other and the information is exchanged between a Trainer's App and Trainee's App; and/or wherein the smart device is at least one of a smart phone, smart watch, smart band, smart glasses, tablet, and computer; and/or wherein the smart fitness device is at least one of a treadmill, an exercise bike, a rowing machine, a weight machine, and a step machine; and/or wherein the Trainer's App displays training-related information of a plurality of Trainees during a training session; and/or wherein the near-range communication is via Bluetooth; and/or wherein the long-range communication is via WiFi.

In yet another aspect of the disclosed embodiments, a method for remote training trainees, utilizing a live trainer is provided, comprising: running an exercise training App on at least one smart fitness device, having at least a near range communication capability and a long-range communication capability, the long-range communication capability providing communication to a network cloud, wherein the App monitors an operational metric of the at least one smart fitness device; running the exercise training App at least one smart device, in proximity to the at least one smart fitness device, having a near range communication capability to the at least one smart fitness device and a long range communication ability to the network cloud; communicating data from the running Apps to a server in a network cloud; a Trainer side system of the at least one smart fitness device and smart device; a Trainee side system of the at least one smart fitness device and smart device; conducting an exercise session on the smart fitness devices; monitoring from the Trainer side system, the Trainee side system's activity; and altering from the Trainer side system, the Trainee side system's smart fitness device operation.

In yet another aspect of the disclosed embodiments, the above method is provided, wherein a user on the Trainee side system can override the Trainer side system's alteration; and/or comprising a plurality of Trainers managing a plurality of remote Trainees, wherein at least one of a given Trainer or Trainee manages a subset of the plurality of Trainees; and/or further comprising, notifying or alerting on the Trainer side system, a non-Trainer designated state of activity by the Trainee.

### BRIEF DESCRIPTION OF THE DRAWINGS

FIG. 1 is a block diagram of a high-level configuration of an exemplary embodiment.

FIG. 2 is a high level block diagram of one possible configuration of hardware to facilitate a Treadly System.

FIG. 3 is an illustration describing a master-slave embodiment.

FIGS. 4A-B are representations of one or many possible displays on a Treadly App for effecting a training session

### DETAILED DESCRIPTION

### Introduction

Several typical fitness training approaches are summarized:

Traditional Model—Trainers work with Trainees in a closed proximity environment (e.g., in a gym). Such an environment allows Trainers to:

a. Monitor the status of the Trainee's progress.

b. Adjust the work out style and/or intensity real-time based on the Trainee's performance.

c. Demonstrate the intended fitness style/movement/routine to the Trainee real-time.

d. Work with a group of Trainees.

“Other” Fitness Training Models include:

a. Offline training—such as playing a pre-recorded Trainer video.

b. Live remote training—such as a video conference session between Trainer and Trainee.

These “Other Models” do not provide the same level of attention and features of Traditional Training, however they provide some unique features:

a. The Trainee can train remotely (e.g., home, office, etc.) and is not “bounded” in close proximity with other Trainees.

b. The Trainee can train at any time and is not limited to the schedule of the Trainer.

However, as more Trainees are looking for remote and available-any-time schedules, the traditional and “other” Training Fitness models are inadequate for their needs.

### Overview

Embodiments of the exemplary invention are now detailed. For ease of reference, an embodiment of the invention will be referred to as a “Treadly System.” An exemplary Treadly System comprises one or more of:

Treadly smart fitness device(s) (e.g., treadmill, exercise bike, rowing machine, a weight machine, and a step machine, pressure sensitive mat, etc.) These fitness devices may have a display as well as speakers and microphones and bio-sensors, if so desired. These fitness devices are capable of communicating to a smart device, such as a smart phone, smart watch, smart band, smart glasses, tablet, computer, etc. or to the Cloud directly. It is understood that the various devices may have microphone(s), speaker(s), display(s), User interfaces, motion detector(s), etc. that are common in the smart device arena;

Treadly Apps (iOS, Android, Computer, etc.) installed on the Treadly fitness device(s) and/or on a user's smart phone or equivalent device, to enable communicating directly or indirectly with the various App-installed device(s) to a Cloud network, for example, the Internet. Further, the App on the Treadly fitness device(s) can (in some instances) enable a degree of control by a Trainer over the Trainee's smart fitness device(s) as well as enable parameters/metrics of the fitness device(s) to be forwarded to the Trainer's App. It is noted that the App on a smart fitness device(s) may be of a different version or type than one that is installed on a smart phone or equivalent device. Nonetheless, these Apps can coordinate between each other and facilitate the desired exercise program; and

Treadly Cloud for communicating with the Treadly Apps running on the smart device(s) and/or on the Treadly fitness device(s) and performing management & coordination among different instances of Treadly Apps(s) and Treadly device(s) (i.e., users). The Cloud includes one or more servers providing the “server-side” management of the data being exchanged between the App platforms. Server implementation of Apps or software on a “client-side” is well understood in the art and is accordingly within the purview of this disclosure.

For a Trainer-Trainee session, each person (trainer and trainee) would have an App running on their smart device and/or fitness device. And communication between each other's device(s) (e.g., fitness device and/or smart device) is facilitated by default via the smart device(s) comm abilities. However, in other embodiments, the exemplary system can have the fitness device(s) communicate directly to the Cloud containing the server running the software for supporting the Apps on the user's smart devices and/or on the fitness device(s). Therefore, the App (on whichever platform) may communicate to the Cloud and the Cloud communicate to the App.

By coordinating a combination of the above features, the Treadly System can facilitate communication between a Treadly fitness device(s) used by a trainee and with a trainer (who can monitor the trainee's progress, bio signals, face, movements, control and adjust the user's Treadly fitness device(s), etc.) in a more precise and efficient manner than previously know.

Specifically, the Treadly System solves the Problem Statement:

a. Provides a Training Session:

b. Allows one user to be the Trainer.

c. Allows multiple users to be the Trainees.

During the Training Session, 1-to-many duplex video/audio sessions can be established. For example, the user's smart device's camera can be used to provide the video feed (or if the Treadly fitness device is camera-enabled, then through the fitness device). Wireless connection to the Treadly Cloud can be similarly (if so configured) handled through the smart device's wireless capabilities.

The Trainer can broadcast his/her video/audio session to multiple Trainee(s). Each Trainee can watch the Trainer's video/audio (on the Treadly fitness device's monitor or/and via the Treadly App's display on the user's smart device). The Trainer's video can be real time or can be pre-recorded, depending on the configuration and arrangement desired.

Trainee(s) can also broadcast his/her video/audio session to Trainer. (Trainer can watch Trainee's video/audio, on his/her Treadly fitness device or/and via the Treadly App's display or a computer, etc.).

During the Training Session, the status from the Treadly fitness device (such as speed, number of steps, etc.) of each Trainee can be sent to the Trainer to allow Trainer-monitoring of each Trainee's status, real-time. Note—Fitness device status information provides accurate unbiased info about the Trainee's training session, real time.

During the Training Session, the Trainer can configure each individual Trainee's fitness device, or a group of Trainees', to match the Trainer's fitness device (if the Trainer is using one), such as speed, foot steps, etc. in real-time, if so desired. Or to a metric that the Trainer deems is appropriate, if the Trainer's video session is pre-recorded. The match factor can be adjusted by the Trainer (in some instances, the Trainee may override the Trainer's setting). Alternatively, the Trainer can send varying target goals to Trainee's fitness device and the Trainee can work to those metrics within a session interval (e.g., target goal is 10% faster speed on stationary bike, within a 30 second window, etc.)

As a specific non-limiting example, Trainer is at 4.0 mph, and Trainer can configure Trainee's fitness device at 100% match (i.e., 4.0 mph) or at 90% match (i.e., 3.6 mph). Trainer can further configure the Trainee's fitness device to match the workout style, such as feet movement, elevation, resistance level, etc.

For example, Trainer has various Stride patterns and width, and would like the Trainee to follow it. Treadly System can provide indication (on fitness device itself, on an App, lighting a fitness belt, sounds, etc.) if Trainee is not matching the workout style configured by the Trainer.

When the Trainer is remote-configuring on the Trainee's fitness device, the real-time feedback from the Trainee's fitness device will be provided back to the Trainer for real-time analysis. Feedback can be immediately made by the Trainer (e.g., heart rate increasing dangerously—Trainer can slow the pace, intensity, etc.). Health monitoring software can also be implemented to assist or indicate to the Trainer when a dangerous or elevated situation is occurring.

The entire Training Session can be recorded and played back (including video/audio and all configurations related to Treadly Systems—e.g., the status from the fitness devices). In some instances, a recorded session can be played to the Trainee during subsequent training session, as a baseline wherein the Trainee can attempt to match or exceed his/her previous performance (presumably under guidance of Trainer). In another instance, the recorded data from the Training Session can be evaluated to provide desired goals for the upcoming Training Session(s) that match a given profile or pattern. That is, trending or other metrics can be evaluated either by the Trainer (or helper to the Trainer) or by a computer software system, e.g., artificial intelligence, etc.)

With the Trainer able to monitor (real-time) and also send adjustments or target metrics to the Trainees, a more customizable and targeted workout can be achieved both for individuals and for groups. For a Trainee group, the Trainer can segregate the group into different tiers and send different adjustments, requirements, goals to different groups, based on how the Trainer evaluates each group's performance. Mapping of each person's performance can be displayed on a large display (typically a computer) or below/exceeding performances can be highlighted via color change, shape change, sound, etc. on a smaller display (typically a smart phone or similar display).

It should be appreciated that with the use of smart devices, the Trainer and/or Trainee can “touch” the smart device's touch sensitive screen (or trigger—perhaps by vocalizing a word) to menu or investigate a Trainee's parameter, or respective icon or image being displayed. As one non-limiting example, a matrix of real-time performance traits for each individual in a training session may be displayed on a screen. Wherein if one individual's performance is sub-par, that individual's parameter/image/etc. may change color or shape, etc. For example, the Trainer may select (e.g., touch) that “individual” to trigger direct the Trainer's voice target to that individual's smart device or Treadly fitness device. Thus, the Trainer may “speak” only to that individual without having to speak to the rest of the group. Or, vice versus. The ability to focus the Trainer's direction to a given Trainee (without other Trainees' knowledge) provides a great degree of “privacy” during the Training Session.

As can be apparent, the exemplary system enables a single Trainer to custom train multiple remote Trainees who themselves may be at different sites (that is, the Trainees do not share the same location).

While the exemplary description above is in the context of a single Trainer training one or more Trainees, it is understood that multiple Trainers may jointly train multiple Trainees. In such a scenario, it may be possible for an assistant Trainer to “take over” for a given sub-group of Trainers, or alternatively, in some embodiments, an advanced Trainee may be given limited Trainer privileges to perhaps lead one or more other Trainees within the Training Session. In this latter scenario, the Treadly System can provide a separate screen image of that proxy Trainer's session (and Trainee group) to the principal Trainer for his/her monitoring.

As evident in the below Figs. and above details, the exemplary system in one or more embodiments provides a new fitness training model with all of the features in Traditional Fitness Models while able to be used in a remote environment and available any time.

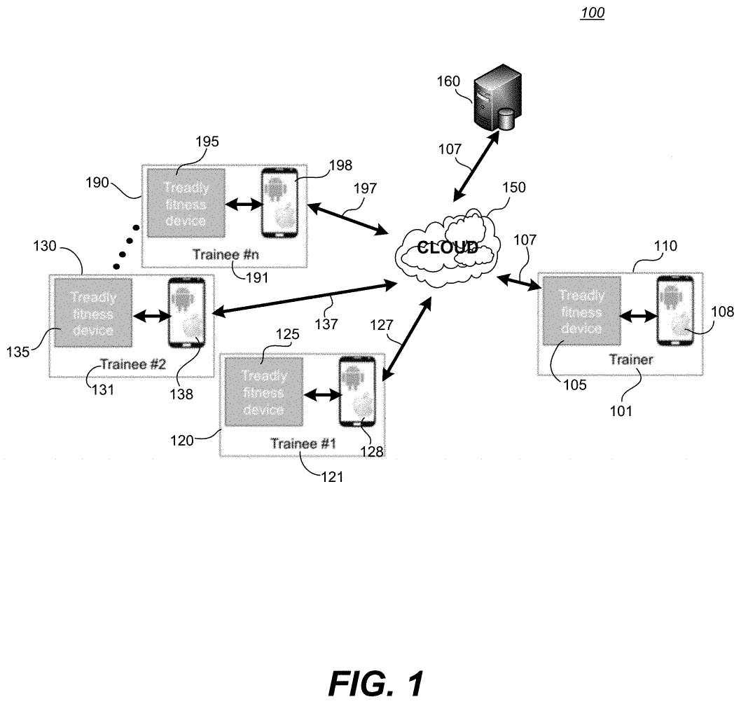

FIG. 1 is a block diagram 100 of a high-level configuration of an exemplary embodiment. Trainer system 110 is composed of one or more Treadly fitness devices 105 (which may be a smart exercise device 105, or a smart bio-monitoring device such as a smart watch, smart band, smart glasses, etc. which communicates, via wireless or wired, to a smart phone 108 or tablet or equivalent. In some instances, the Treadly fitness device 105 may incorporate one or more of the necessary features of the smart phone 108, thereby obviating specific needs from the smart phone. In some embodiments, the Treadly fitness device(s) 105 may be unnecessary if the training session is one of aerobics, or the like, where a so-called fitness machine is not necessary. In this scenario, the smart device (for example, smart phone), running the Treadly App, will provide the needed communication and feedback resources.

Trainer 101 is operating the Trainer system 110. The Trainer system 110 communicates to one or more remote Trainee systems 120, 130, 190 via wireless links 107 to a Cloud 150 having Treadly server 160 processing and managing information exchanged between the Trainer's and Trainee's systems 120, 130, and 190. On the client-side/Trainee-side, communication links 127, 137, 197 enable data from Trainee systems 120, 130, and 190 to be sent/received to the Treadly server 160 via the Cloud 150. In some embodiments, there may be only one link to a master Trainee device (being either a Treadly fitness device or Trainee's smart “phone”) having other Trainee devices in a slave mode, if so desired. In such or different configurations, links 127, 137, 197 may also forward data from respective Trainee devices, that is, the Trainee devices may communicate to each other.

Each Trainee system 120, 130, 190 contains Treadly fitness device(s) 125, 135, and 195, which may be a smart exercise or Trainer-equivalent or nearly equivalent fitness device 105, or a smart bio-monitoring device such as a smart watch, smart band, smart glasses, etc. which communicates, via wireless or wired, to respective smart phones 128, 138, and 198, or tablet or equivalent. Each Trainee system 120, 130, 190 is operated by a respective Trainee 121, 131, 191.

Each Treadly fitness device 120, 130, 190 and/or smart “phone” 128, 138, 198 contain a Treadly App or program that coordinates communication and monitoring of the training session to the Cloud. Utilization of the embodiments shown above can be according to any of the approaches and methods described in the above Overview.

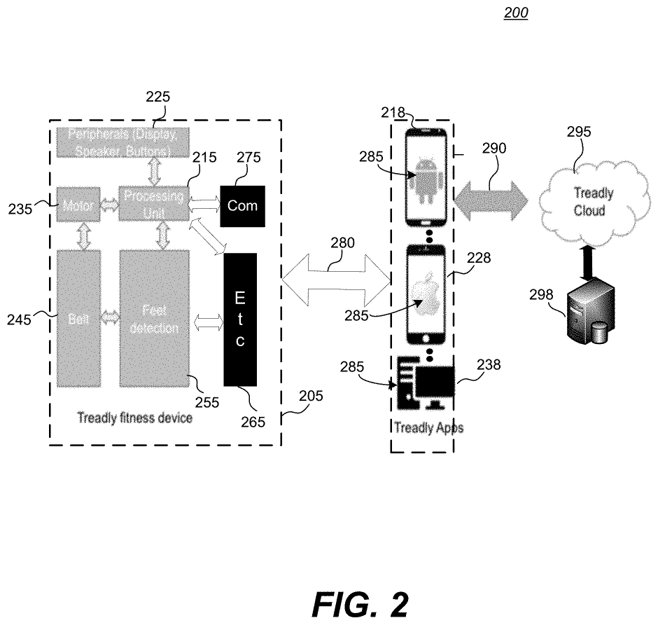

FIG. 2 is a high level block diagram 200 of one possible configuration of hardware to facilitate a Treadly System. Here, the example is for a treadmill like fitness device. Of course, other fitness devices that are not treadmill based may be similarly utilized. In this example, Treadly fitness device 205 comprises a “smart” fitness machine with a processor 215 with appropriate peripherals 225 for user interaction (e.g., display, buttons, speaker, etc.), motor(s) 235 that drives a belt 245 about foot detector(s) 255, and other sensor(s) 265 (e.g., hand grip for pulse, etc.). In some embodiments, the belt 245 may sit on top of the foot detector(s) 255. Processor 215 provides external communication via COM module 275, which may be via a wired or wireless connection 280. If communicating to a local device, a near range communication protocol such as Bluetooth® (trademark registered by Bluetooth Special Interest Group) may be utilized. If communicating long range directly to the Cloud, an Internet based communication may be utilized as well as using WiFi if appropriate.

The User's smart device 218, 228, 238 receives data from the Treadly fitness device 205, wherein the smart device 218, 228, 238 is running a Treadly App (or software) 285. The App 285 manages the data and interfaces with the User (not shown) in whatever capacity the User is (e.g., Trainer, Trainee). Appropriate smart device-processed data is forwarded 290 to the Treadly Cloud 295 for operation by a Treadly Server 298 embedded in the Cloud 295. The Treadly Server 298, in return, transmits its operated data to the App 285 on the respective smart devices and fitness devices, for displaying information, communication, controlling, etc. in the context of the Training session.

It is expressly understood that there can be an embodiment where the Treadly smart fitness device 205 is a smart fitness device, but not Treadly-configured. That is, it is not capable of running a Treadly App or being controlled by such. In this situation, the smart fitness device will likely have a data output port (wired or wireless), wherein the output data is sent to the Trainee's smart phone (or equivalent device) running the Treadly App. The App may be configured to recognize a non-Treadly fitness device and act appropriately, forwarding the information to the Cloud/Trainer's App. In this instance, the Trainee's fitness device will not be controlled by the Trainer. Utilization of the embodiments shown above can be according to any of the approaches and methods described in the above.

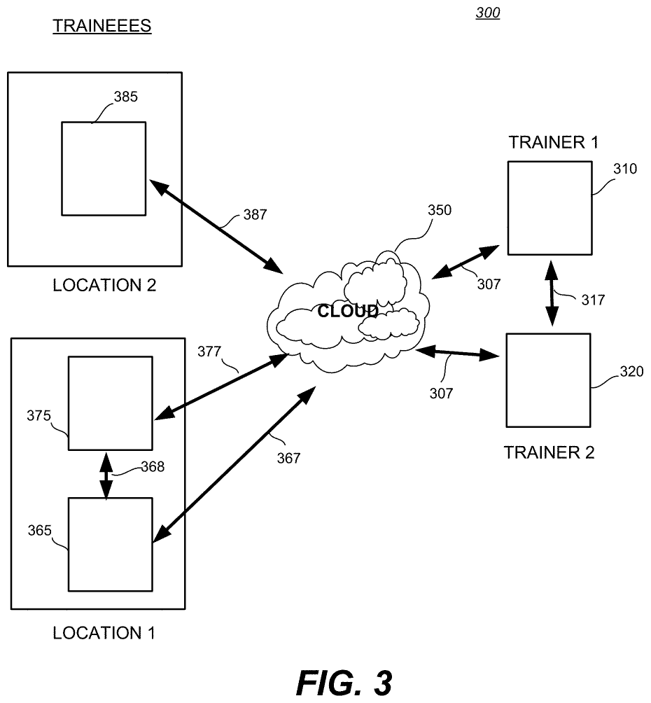

FIG. 3 is an illustration 300 describing a master-slave embodiment. Trainer 1 (310) with Trainer 2 (320) may be jointly conducting a training session with remote Trainees 365, 375, 385, with Trainees 365 and 375 sharing a common location, for example, while Trainee 385 is at a different location. Similarly, Trainers 1 and 2 may be at different locations. Communication between the Trainers is facilitated through link 317 while each Trainer may communicate to the Cloud (having the Treadly server—not shown) 350 via one or more links 307. Trainer 1 (310) may be the primary trainer while Trainer 2 (320) can be the secondary trainer or vice versus. In any event, one trainer may be the “master” while the other may be the “slave,” if so desired. Communication between the Trainers and Cloud 350 and the respective Trainees' systems are facilitated through links 307, 367, 377, 387, which may also be master-slave coordinated. A master-slave arrangement for communication may happen at the trainee site, for example, Trainee 365 may be the master link for Trainee 375, handling communication from the Cloud 350 and passing to Trainee 375 via link 369. Further, Trainer 1 (310) or Trainer 2 (320) may be dedicated (or shift) to one or more of the trainees (either co- or not co-located) in case one group of trainees need special attention, thus allowing the session to progress without interruption. Further, as stated above, a trainee may act as a proxy trainer, depending on the training parameters set by a Trainer. Utilization of the embodiment shown above can be according to any of the approaches and methods described in the above.

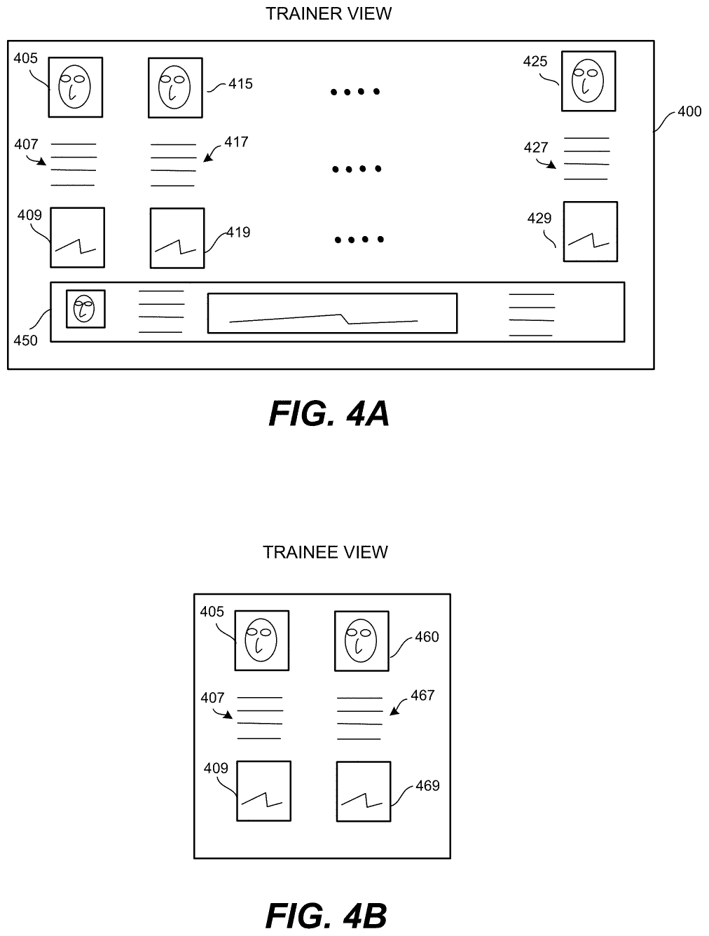

FIGS. 4A-B are representations of one or many possible displays on a Treadly App for effecting a training session. It is expressly understood that the appearance and functionalities of these representations can be easily modified and changed by one of ordinary skill, according to design preference and, therefore alternate embodiments and configurations are understood to be within the scope of this disclosure. The screen displayed may be on the Treadly smart fitness device or smart phone (or equivalent).

FIG. 4A illustrates a possible Trainer screen 400, with video/camera images or representations of one or more Trainees 405, 415, 425. Associated with each Trainee is a training session parameter/metric outputs 407, 417, 427 showing current bio/performance readings of the respective Trainee. Optionally, a chart or plot 409, 419, 429 of one or more bio/performance metrics can be displayed, according to design preference. These outputs or metrics can be of the Trainee's physical condition as well as the smart fitness system of the Trainee. As a reference, the Trainer's own body and system's metrics 450 are displayed. In some instances, an alert may be sent to the Trainer for a non-Trainer designated state of activity by the Trainee.

FIG. 4B illustrates a possible Trainee screen 470, with video/camera images or representations of himself 405 and Trainer 460. As in FIG. 4A, current bio/performance readings 407 of the Trainee and Trainer 460. Optionally, a respective chart or plot 409, 469 of one or more bio/performance metrics can be displayed, according to design preference. Utilization of the embodiment shown above can be according to any of the approaches and methods described in the above.

Accordingly, as will be appreciated by one skilled in the art, the present disclosure's descriptions of hardware may be embodied as an apparatus that incorporates some software components. Accordingly, some embodiments of the present disclosure, or portions thereof, may combine one or more hardware components such as microprocessors, microcontrollers, radio modules, or digital sequential logic, etc., such as processor with one or more software components (e.g., program code, firmware, resident software, micro-code, etc.) stored in a tangible computer-readable memory device such as a tangible computer memory device, that in combination form a specifically configured apparatus that performs the functions as described herein. These combinations that form specially-programmed devices may be generally referred to herein as “modules”. The software component portions of the modules may be written in any computer language and may be a portion of a monolithic code base, or may be developed in more discrete code portions such as is typical in object-oriented computer languages. In addition, the modules may be distributed across a plurality of computer platforms, servers, terminals, and the like. A given module may even be implemented such that the described functions are performed by separate processors and/or computing hardware platforms.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. A system for remote training trainees, utilizing a live trainer comprising:
at least one smart fitness device, having at least a near range communication capability and a long-range communication capability, the long-range communication capability providing communication to a network cloud;
at least one smart device, in proximity to the at least one smart fitness device, having a near range communication capability to the at least one smart fitness device and a long range communication ability to the network cloud;
an exercise management App running on the at least one smart fitness device and smart device; and
a server in the network Cloud, communicating with the App and managing other Apps on the at least one smart fitness device and smart device;
wherein a Trainer using the App on his at least one smart fitness device and smart device is able to real-time monitor a metric of a remote Trainee training on her smart fitness device via the Trainee's smart fitness device's App or the Trainee's smart device's App, and alter an operational parameter of the Trainee's smart fitness device.

2. The system of claim 1, wherein at least one of the smart fitness device(s) and smart device(s) communicates information to each other and the information is exchanged between a Trainer's App and Trainee's App.

3. The system of claim 1, wherein the smart device is at least one of a smart phone, smart watch, smart band, smart glasses, tablet, and computer.

4. The system of claim 1, wherein the smart fitness device is at least one of a treadmill, an exercise bike, a rowing machine, a weight machine, and a step machine.

5. The system of claim 1, wherein the Trainer's App displays training-related information of a plurality of Trainees during a training session.

6. The system of claim 1, wherein the near-range communication is via Bluetooth.

7. The system of claim 1, wherein the long-range communication is via WiFi.

8. A method for remote training trainees, utilizing a live trainer comprising:
running an exercise training App on at least one smart fitness device, having at least a near range communication capability and a long-range communication capability, the long-range communication capability providing communication to a network cloud, wherein the App monitors an operational metric of the at least one smart fitness device;
running the exercise training App at least one smart device, in proximity to the at least one smart fitness device, having a near range communication capability to the at least one smart fitness device and a long range communication ability to the network cloud;
communicating data from the running Apps to a server in a network cloud;
a Trainer side system of the at least one smart fitness device and smart device;
a Trainee side system of the at least one smart fitness device and smart device;
conducting an exercise session on the smart fitness devices;
monitoring from the Trainer side system, the Trainee side system's activity; and
altering from the Trainer side system, the Trainee side system's smart fitness device operation.

9. The method of claim 8, wherein a user on the Trainee side system can override the Trainer side system's alteration.

10. The method of claim 8, comprising a plurality of Trainers managing a plurality of remote Trainees, wherein at least one of a given Trainer or Trainee manages a subset of the plurality of Trainees.

11. The method of claim 8, further comprising, notifying or alerting on the Trainer side system, a non-Trainer designated state of activity by the Trainee.
